(19) United States
(12) Patent Application Publication
(10) Pub. No.: US 2005/0039127 A1
(43) Pub. Date: Feb. 17, 2005

(54) Electronic navigation of information associated with parts of a living body

(75) Inventor: Mark Davis, Newton, MA (US)

Correspondence Address: FISH & RICHARDSON PC, 225 FRANKLIN ST, BOSTON, MA, 02110, US

(73) Assignee: Haltsymptoms.com, Inc., a Massachusetts corporation

(21) Appl. No.: 10/666,112

(22) Filed: Sep. 18, 2003

Related U.S. Application Data
(63) Continuation of application No. 09/690,625, filed on Oct. 17, 2000, now abandoned.
Application No. 09/690,625 is a continuation-in-part of application No. 09/615,884, filed on Jul. 14, 2000, now abandoned.

(51) Int. Cl. A61B 5/00; G06K 9/00; G06F 17/60; G09G 5/00

(52) U.S. Cl. 715/708; 715/835; 715/810; 715/764; 715/705; 715/706; 715/709; 715/710; 715/711; 715/712; 715/713; 715/714; 715/715; 600/300; 600/301; 382/128; 705/3; 705/2

(57) ABSTRACT

A stylized graphical image of a human body is displayed on a mobile computer, cell phone or other device. The user selects a body part of interest in a standard manner as dictated by the host platform. In response to the user selecting a body part, information is provided that is associated with physical aspects of the selected body part including symptoms and medical conditions. A wide variety of uses are possible with the system. The business model on which the system is commercialized provides benefits to medical providers, drug and product manufacturers, advertisers, and users.

This is a continuation-in-part of U.S. patent application Ser. No. 09/615,884, filed Jul. 14, 2000.

### BACKGROUND

This invention relates to electronic navigation of information associated with parts of a living body.

Information about, for example, symptoms resulting from and/or affecting parts of the human body (and about products that are useful with respect to those parts) is available in print, on CD-ROM, and through networks such as the World Wide Web.

A user can find information in these media using navigation tools such as indexes and tables of contents (in printed materials) and hyperlinks (on the web).

Symptom-based approaches have been used for organizing and presenting medical information.

### SUMMARY

In general, in one aspect, the invention features (a) displaying a graphical image of a living body, (b) enabling a user to select a part of the body that is of interest to the user by pointing to the part of the body on the graphical image, and (c) in response to the user selecting the part of the body, providing information associated with physical aspects of the selected body part.

Implementations of the invention may include one or more of the following features. The graphical image may include a stylized image of a human body. The user may select the body part by having the system alter the graphical image (e.g., by displaying a legend or by changing a visible attribute of the body part) in response to the user moving a pointer over the body part. The graphical image may include another living body of a different age or sex.

The information that is provided may include a list of symptoms or other information related to the body part or a list of products for treating the body part or a mechanism for buying each of the products. The user may select among symptoms and be provided with information about diagnoses and other information in response to his selection.

An icon may be displayed that is representative of the graphical image, and the graphical image of the living body may be displayed in response to the user invoking the icon.

The features of the invention may be implemented on an electronic network such as the World Wide Web.

Among the advantages of the invention are that users have direct, simple, fast, and intuitive access to medical information, product information and purchase opportunities, and medical specialty appointment scheduling and referrals using graphical representations of the human body. In addition, physicians may use the tool to assist in documenting patient information and in the ordering of therapeutics.

Other advantages and features will become apparent from the following description and from the claims.

### DESCRIPTION

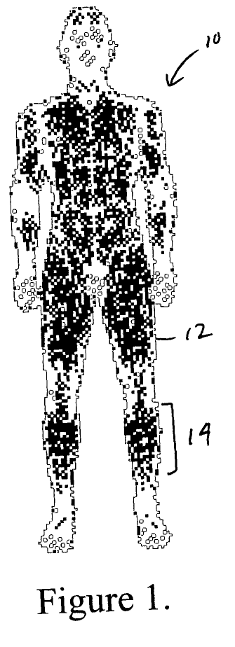

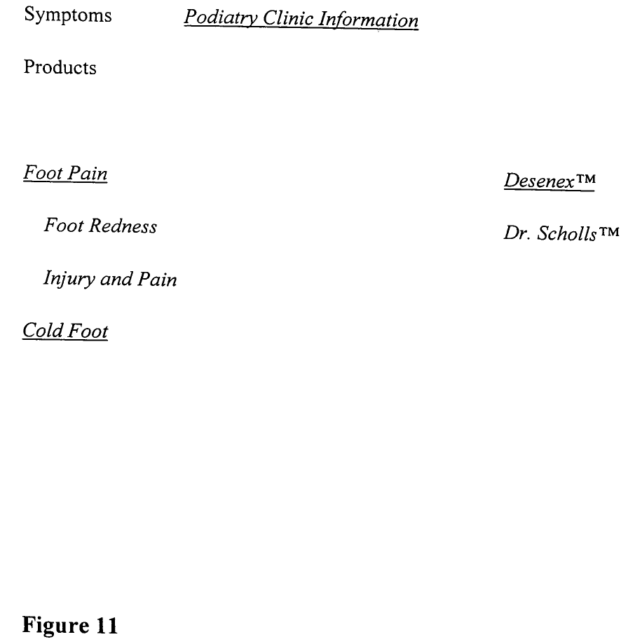

FIGS. 1 through 11 illustrate implementation aspects of the invention that are discussed below.

As shown in FIG. 1, a graphical device that is useful for navigating a web site for information associated with parts of a living body is a stylized graphical image 10 of a human body. The image is formed of relatively low resolution gray scale (shown) or color pixels 12 that impart to the viewer a sense of the three-dimensionality of the body and also visually imply the locations and configurations of different parts of the body, such as the lower leg 14.

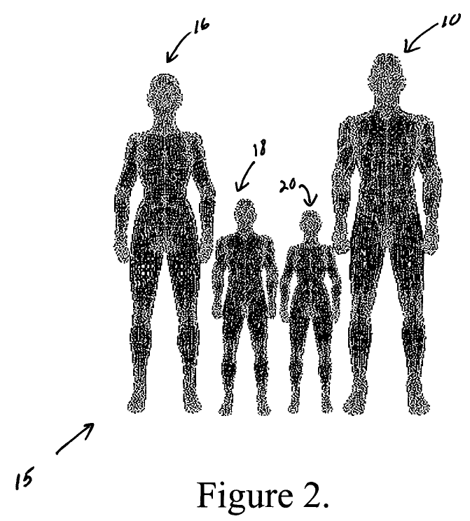

FIG. 2 shows the male graphical image 10 in a grouping 15 with similar figures representing a female human adult body 16, a male child 18, and a female child 20.

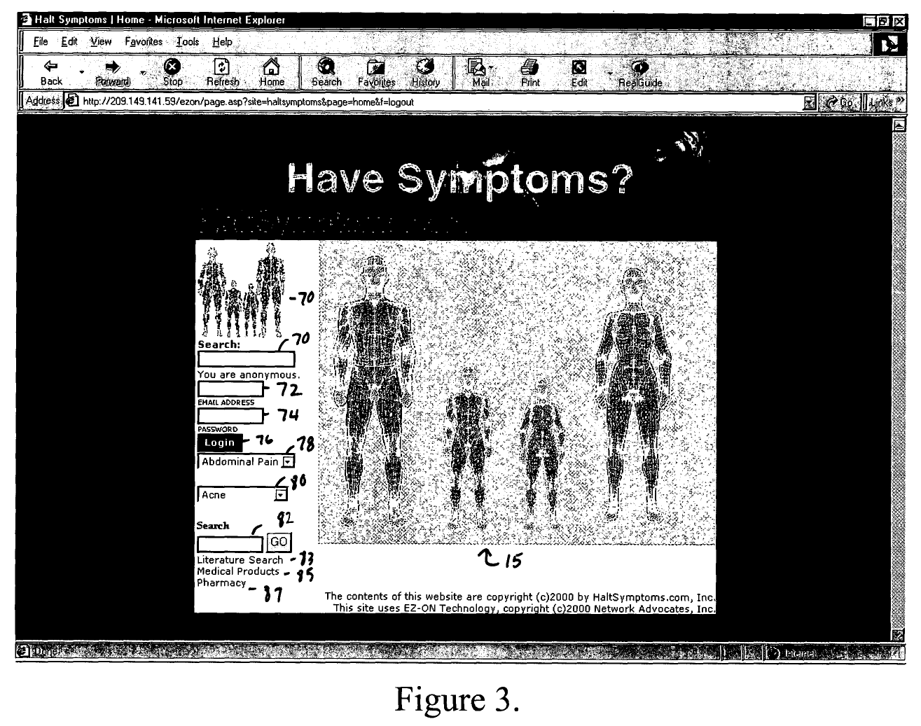

In FIG. 3, the grouping 15 is displayed (with the positions of the male and female bodies reversed) on a web page 30.

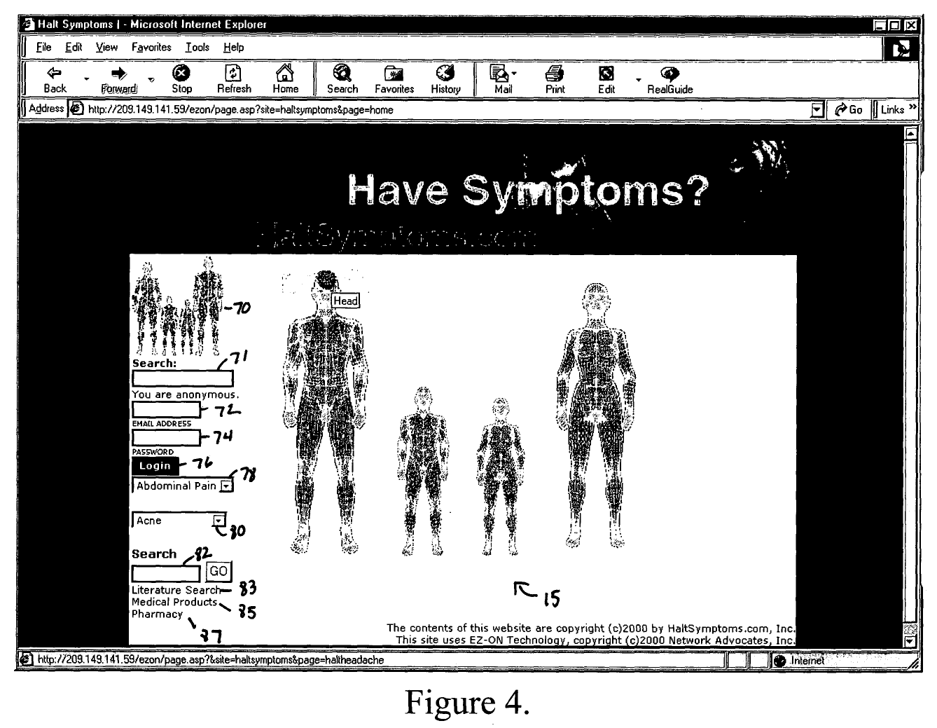

As shown in FIG. 4, each displayed part (segment) of each of the graphical images in the grouping 30 is programmed to exhibit a “hot” spot that is triggered whenever the user moves a pointer (not shown) over that part of the body. For example, when the user mouse-rolls the pointer over the head of the male body, the hot spot causes the legend “HEAD” to be displayed and also causes the color 33 of the head to change to confirm visually to the user the part of the body that is being identified.

The parts of the body for which hot spots are provided are chosen to reflect the parts of the body for which a user typically may have symptoms, complaints, interests, or needs. Each part (segment) is also chosen to be large enough on the displayed graphical image to permit easy identification and clicking as the pointer is rolled over it but not so large as to span parts of the body for which symptoms and products are unrelated.

Examples of suitable body segments include eyes, top of head, ears, chest, abdomen, arms, hands, legs, and feet.

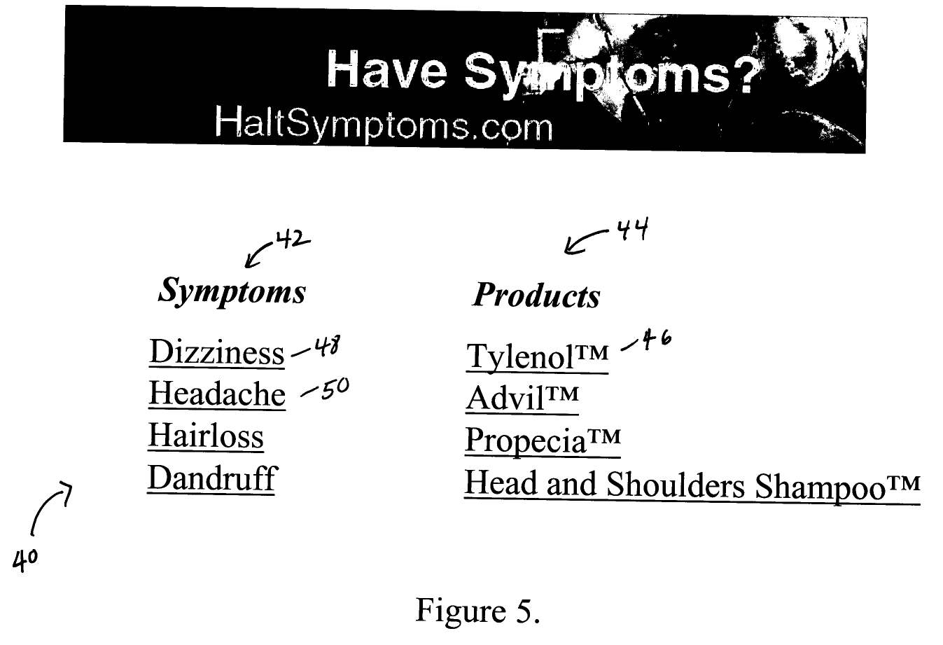

By left clicking when the word HEAD is displayed, the user links to other information stored on a web server as shown in FIG. 5. The other information is presented in the form of a web page 40. The information on web page 40 is organized in two columns representing SYMPTOMS 42 that occur in the male head and PRODUCTS 44 that are useful for the male head.

Each of the products and symptoms is displayed as a hot link.

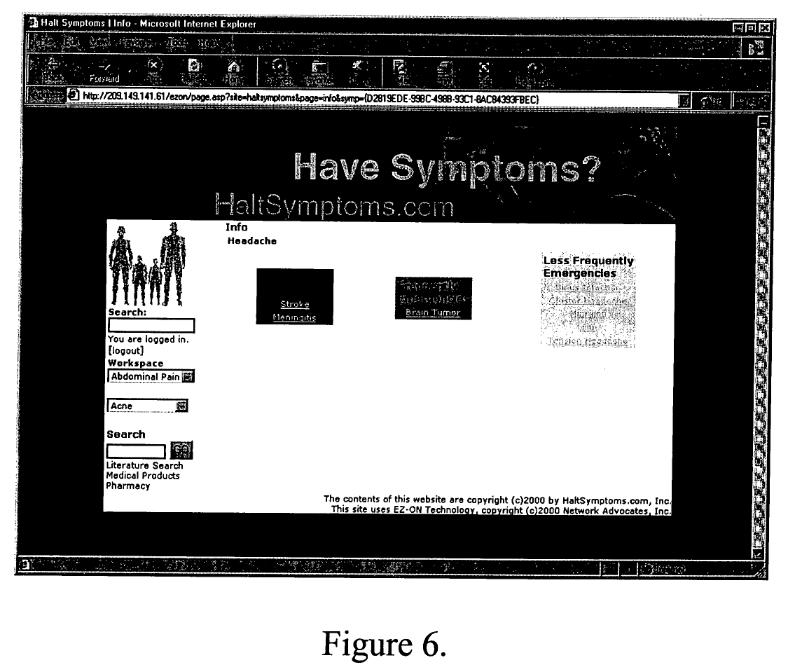

Clicking one of the symptoms, such as HEADACHE 50, will invoke additional information, for example, a list of diagnoses that relate to the headache symptom, as shown in FIG. 6. In this example, diagnoses are divided into categories representing emergencies, frequently occurring emergencies, and less frequently occurring emergencies. Clicking one of the products, such as Propecia™, will, for example, display a page on which the product may be purchased or may invoke the web site of the manufacturer of that product.

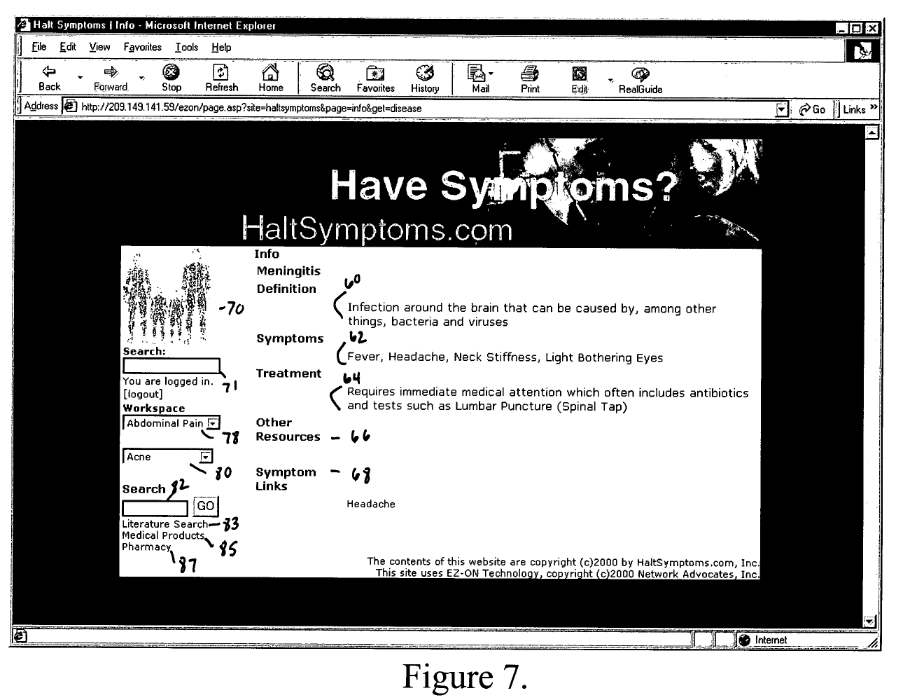

In FIG. 6, if the user clicks on the diagnosis MENINGITIS, he is presented with the web page shown in FIG. 7, which contains information about meningitis, including a definition 60, symptoms 62, treatment 64, other resources 66 (none shown in this example), and symptom links 68. FIG. 6 may represent a separate web site that is devoted to a particular symptom such as headache. FIG. 7 may represent a separate web site that is devoted to a particular disease such as meningitis.

The web pages shown in FIGS. 3, 4, and 7 also provide boxes in which a user can enter a search 71. He can also use drop down lists 78, 80 to identify symptoms and diagnoses. A search box 82 enables searching for literature and medical products. Boxes for an email address 72 and a password 74 are provided for administrator access to the site and are not shown to the users of the system. The site search function allows user specified searching of the HaltSymptoms.com network. The Symptom drop down menu provides direct user access to symptom based medical information (e.g. HaltHeadache.com, FIG. 6). The Disease drop down menu provides direct user access to disease-based medical information (e.g. HaltMeningitis.com, FIG. 7). The literature search function provides users access to medical research and writings. The medical products link provides product purchase opportunities. Items 83, 85, 87 provide direct hotlinks from literature search to literature, medical products to products and pharmacy to pharmacy without use of the search box 82.

A user may invoke the nagivation capability of the graphically displayed group 15 either by using his browser to access a web site that directly provides the navigation image as a primary element (as in FIG. 3) or a web site that displays the navigation image in the form of an icon 70 (FIG. 7) as an adjunct to other features. In the latter case, when the user clicks on the icon 70, he is transferred to the site or web page that provides the full navigation graphical image.

The user could be led to either type of web site by typing the URL or through search engines or other methods of referral.

In one example of a use of the invention, a user would go to a site at URL www.HaltSymptoms.com of the kind illustrated in FIG. 3. If the user sought information about headache, he would move his mouse pointer over the top of the head and then select HEAD (FIG. 4). He would then be presented with FIG. 5, would select Headache, and would be taken to www.HaltHeadache.com (FIG. 6), an area with medical information which includes the diagnoses which might be causing the headache. The user might choose to click on “Meningitis” which is a medical emergency and be given information regarding this lifethreatening condition (FIG. 7). The graphical image icon 70 will be available in the left side bar which, when clicked, will bring back the full graphical image for use in navigation.

In another example, a user would go to www.HaltHeadache.com (FIG. 6) after being directed to that site by an advertisement on another site. The user might choose to get information regarding the diagnosis of Migraine that includes a definition, symptoms, treatment, other resources, symptom links, and sponsors. In the left sidebar the icon 70 is available to activate the full graphical image 15. The user might use the mouse to point at the icon after getting the information he desires regarding headache. For example, he might point the mouse arrow to the neck because in association with the headache, he wishes to learn about neck pain. The neck will become highlighted and change color, and the word “neck” will appear. When the mouse click is performed over the neck, the page with symptoms and products related to the neck will appear.

In a third example, the user goes to www.HaltAbdominalPain.com and finds information about Peptic Ulcer Disease. In this section, the user is informed that upper abdominal pain might also indicate a heart attack. The user mouse clicks over the diagnosis of heart attack to move to the Heart Attack section of www.ChestPain.com. The user then decides to get more information regarding arm pain. He may move the mouse and arrow to the left sidebar where the icon 70 is located. The user might choose to mouse click the icon to open the graphical device 15. The user might then mouse-over the representation of the arm to access conditions associated with arm pain and medical information as this can also be associated with a heart attack.

In a fourth example, a user might wish to order incontinence products through the Internet in the privacy of his home. He arrives at HaltSymptoms.com and points to the pelvic area of the male body graphical image shown in the grouping 15. The pelvic area will change color and the word GENITALS will appear. When the user clicks, he will be brought to a Symptoms and Products list associated with this body part. The user might then order adult incontinence products.

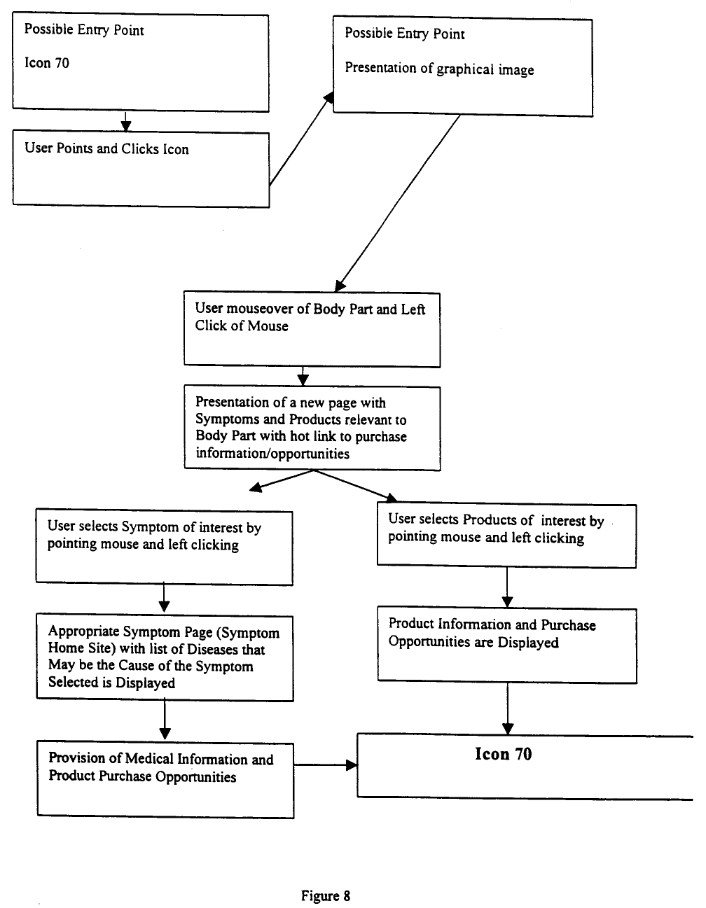

A flow chart of the steps in using the invention is shown in FIG. 8.

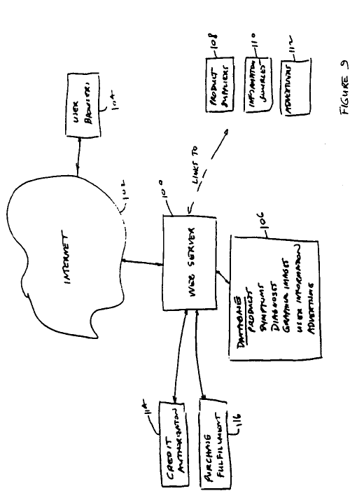

FIG. 9 shows the structural relationships among the web server 100 and other participants. The web server maintains databases 106 that include information about symptoms, diagnoses, products, graphical images, user information, and advertising that may be included on web pages associated with the graphical image nagivation device 15. The web server can provide links to product suppliers 108, information sources 110, and advertisers 112. The web server also interacts with credit authorization servers 114 and purchase fulfillment servers 116 for the purpose of consummating product purchases made by users. The server 100 interacts with user browsers 104 through the Internet 102.

Hardware and Software Implementation Example

In one implementation approach, the web server is an IBM Netfinity 5500 M10 with dual Pentium II Xeon 450 MHz processors, 1 Gb of RAM and three 10,000-rpm 9.1 Gb Seagate Barracuda Ultra-SCSI hard drives in a Level 5 RAID configuration. The software platform is Microsoft Windows NT 4.0 Server running Service Pack 5; Microsoft Internet Information Server 4.0; and Microsoft SQL Server 7.0 with service pack 1.

Other Implementations

Other implementations are within the scope of the following claims.

For example, a variety of other graphical images could replace the group 15, including individual male, female, and child images (not shown in a group), images of parts of the body (such as the chest), and images of non-human living bodies, such as dogs or horses.

Clicking each of the hot spots (such as a hand) of an initial graphical display, such as the grouping of bodies 15, could lead to another more detailed graphical display, such as a display of a hand, in which hot spots would be programmed for segments of the hand, such as the fingers, the finger nails. Additional, more detailed levels of an image hierarchy could also be provided.

The graphical image of the living body need not be stylized or of low resolution. A photographic image could be used.

The navigation need not be done on a web site but could be done on a variety of network configurations or on a variety of media.

The information that is made available to a user when he invokes a hot spot could include specialty sites within a network of a medical publisher relating to, for example, cardiology.

The invention may be made available to a user through a wide variety of stationary and mobile devices. The mobile devices may include portable computers, hand held computing devices (e.g., the 3Com Palm Pilot™), cellular telephones or a specially designed HaltSymptoms device. The method by which the user selects a desired body part within the graphical tool will depend on the capabilities of the host device. Methods of selection may include tapping, mouse-over, highlighting, visual fixation, keystrokes, voice, for example.

The medical information that is to be navigated may be stored on and served from a central server on the World Wide Web or from a local device, a local server, or any other storage/server equipment.

For example, a user might use the stylus of a Palm Pilot to tap on an icon representation of the system to activate the graphical tool. If the Palm Pilot is connected to the World Wide Web through a modem or other device, the user may utilize the Halt Symptoms system in the manner described earlier. Pages served by web sites may be specifically formatted using Wireless Access Protocols (WAP) or other specific formatting to facilitate information exchange with the local hand-held device. The invention and/or information may be formatted specifically for use on a specific device such as the Palm Operating System (OS) for the Palm device.

The availability of the system on a mobile device could enable a variety of uses. For example a consumer who has a cough and is in a drug store could get information about coughs and medications used for coughs. The consumer could access the navigation tool and point to the chest on the adult female (FIG. 2) to access the World Wide Web or other database. The user might be directed to www.haltcough.com or another site on the HaltSymptoms.com, other sites on the Internet, or to locally stored information. The user could then get information about influenza and medications that might be used to treat it. The user could subsequently scan a bar code located on the box of a cough medication, for example, if the hand-held device included an infrared scanner. The consumer might then compare the scanned drug's actions, side effects, and costs with information obtained on the World Wide Web or locally stored database using the system.

In another example, a patient might have an aggravation of his chronic headaches and use the system on a home computer in the manner described earlier.

After mouse over and click of the adult male head (FIG. 4), the user would be presented with a page that might include symptoms and products related to the head body part (FIG. 5). He could choose headache and be presented with information and possible diagnoses (FIG. 6). He might learn that one possible cause for his intermittent left sided head pain is migraine headaches and that a new drug is available that produces fewer life-threatening side effects than the one he is currently using. He then might choose to download the article and save it to his computer possibly with an electronic coupon provided by the site. He might then decide to transfer the article to his handheld computer, phone, or other device. Later, in the physician's office, the patient might display the article to the doctor. The doctor could choose to activate the system to display prescribing information, coupon or other relevant information for the drug or condition.

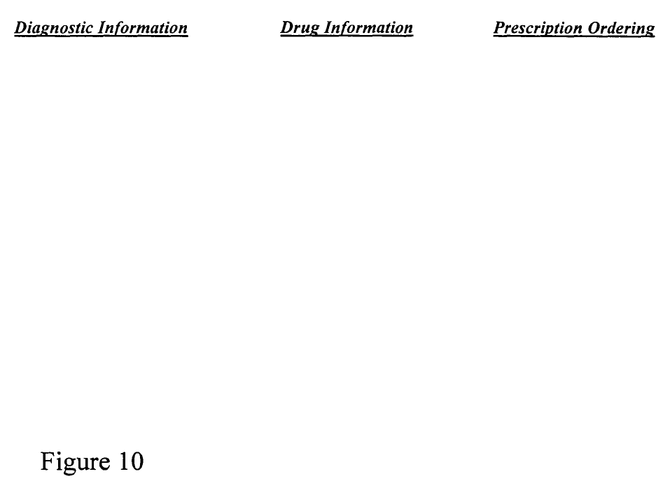

In another example, a patient might bring a mobile phone with Internet access to a physician visit. In the waiting room, the patient might note that his/her knee has become swollen in an area of pain for which he came to the doctor. He might activate his World Wide Web connection and access www.HaltSymptoms.com. He might mouse over the knee and learn that one possible cause for his chronic pain is “septic arthritis” (knee joint infection). He may then discuss this possibility with his doctor who may use the tool to obtain further information. If the swelling is chronic, the patient might get information on degenerative arthritis (overuse arthritis) and learn that there is a new drug available with fewer life-threatening bleeding side effects than the one he is currently using. The patient might choose to present the information on the handheld device to the doctor during the visit. The doctor might agree to have the prescribing information and/or coupon and/or other information transferred to his handheld device by infrared transfer or other means. The doctor could write an electronic order on the handheld device for a variety of patient and/or physician needs including a prescription for the drug transferred by the patient. The doctor might also write an order for other prescription or non-prescription drugs, medical devices or other products and medical tests and send it through his own handheld device, desktop computer or other method through a local network, the world wide web or other network to the appropriate facility such as a pharmacy or other entity for drug dispensing, medical laboratory, or medical supply house. In one application for physicians, a physician using a Palm Pilot or other handheld device might be seeing a patient with abdominal pain. The physician might wish to prescribe medication such as Prilosec, which treats ulcer disease. The physician might tap on the abdomen shown on the handheld device and then be presented with a screen where he chooses among diagnostic information, drug information, and access to direct electronic prescription ordering (FIG. 10). After filling the necessary information, prescription information is sent by modem or direct connection for pharmacy fulfillment.

In another application, physicians using an electronic medical record may wish to record findings regarding a specific body part. A physician might examine a patient in his office and note an irregular sound in the heart. He might activate the system and select the heart. He will subsequently indicate the condition on the hand held device that will become part of the patient's medical record. The physician might follow a similar process by recording medical information into other devices such as a desktop computer using the system.

In another application, a user may be presented with the system while visiting a website belonging to a healthcare maintenance organization. If he had an ingrown toenail he might mouse over the foot and be presented with a symptom list, a link to podiatry appointment scheduling, and purchase opportunities (FIG. 10).

Business Model

An example of a business model that can be built on the system described above establishes brand identity through a network of individually registered, but linked, symptom and disease-based Internet medical sites. Under this model, healthcare manufacturers obtain a unique marketing mechanism through symptom and disease site sponsorships that include advertising rights for specific symptom and disease dot corn addresses. Healthcare sales organizations can acquire a captive consumer base with a particular healthcare need. Healthcare delivery organizations and insurers can get a unique method for providing healthcare information to their constituents in partnership with other industries. Publishers can organize information in a unique consumer-friendly manner. Consumers obtain a unique approach to the highest quality health information.

HaltSymptoms.com is a collection of linked individual URL web sites that share a common patient-oriented templated approach to medical problems based upon symptoms as well as diseases. Patients access the site either through the general URL (www.HaltSymptoms.com) or through any of the registered Halt sites (e.g. HaltBackpain.com). Each Symptom Site provides a unique opportunity for levels of sponsorship by industry including sole site sponsorship. For example, the manufacturer of a headache medication might wish to assume full sponsorship for the HaltHeadache.com site that would allow it to use this name in its marketing campaigns with links to product information/sales opportunities.

Site design and implementation combine principles of current medical thinking with a unique approach to medical education that provides medical information in a manner that is intuitive for non-physicians.

If users access the network through the Central Home Site (HaltSymptoms.com) they will find a page simply organized and designed to be pleasing and promote ease of use and rapid navigation. Users will be quickly taken to their chosen symptom homepage with one click of the mouse through the graphical navigation tool or traditional pull-down menus. A similar organization is maintained at all symptom and disease home sites allowing for easy navigation. The left sidebar will include click through opportunities for Medical Literature Searches, Medical Organizations, Pharmacy Orders, Medical Product Orders, etc. Each symptom home page will include multiple possible diagnoses. Each diagnosis (i.e. disease) will include: a brief description of the condition; symptoms; treatments; other resources; and sponsors (unless the entire symptom URL has been sponsored). The left bar may include one clickable area of medical referrals (HMOS, etc), other advertising and Healthcare sales.

Brand Identity

The use of a series of Halt______.com sites creates brand identity, marketing opportunity, and ease of general use. This unique approach allows corporate partners/sponsors to utilize a specific domain name owned by HaltSymptoms.com (e.g. HaltHeartburn.com) under licensing agreements. These agreements may include the sole rights to advertise using that specific symptom-based domain name which will be a part of the overall network of HaltSymptoms.com.

Brand identity is strengthened as the companies that wish to advertise to people with specific medical complaints (e.g. HaltHeadache.com) utilize this system. For example, pharmaceutical companies interested in educating consumers about prescription and/or non-prescription drugs will be able to provide an easy to remember symptom or disease-based www address in their marketing efforts as they work to educate consumers about treatment alternatives (e.g., www.HaltBackpain.com for the new cox-2 inhibitor pain medications). Likewise manufacturers of devices relevant to a specific symptom or disease can utilize the system.

The HaltSymptom.com program allows advertisers to refer consumers for detailed disease and sponsor product information (possibly with coupon) using an internet name related to the patient's current concern (e.g. HaltFlu.com). Companies may then be more general in their TV, radio, and print campaigns, leaving the details to a more suitable and cost-effective medium.

Halt Symptoms.com by way of its ownership of a sizeable list of domain names that are symptom, condition, and action oriented achieves considerable value and unique positioning because of the value of a common language symptom site network (“megasite”); partnership with industry through sponsorships of patient symptoms sites that include multiple diseases, the branding opportunity through consistent, high quality, and easily readable consumer-ready information, partnership with industry through sponsorships of particular disease sites, partnership with industry through direct sales based upon symptoms sites, and partnership with industry through direct sales based upon disease sites.

1-30. (cancelled)

31. A method comprising
registering a set of Internet domain names containing some words in common and some different words related to health care,
licensing authorizing the use of the different independently registered domain names respectively to by different health-related enterprises, the enterprises including at least one of the following: a healthcare sales organization, a healthcare delivery organization, and a healthcare manufacturer, the domain names containing at least one sequence of at least one character in common and respectively containing at least one sequence of at least one character that is different between domain names, and
establishing at least some common user interface appearance elements for web sites associated with the different respective domain names, and
marketing the different domain names for use by consumers in a way that generates strong common brand identity among them.

32. The method of claim 31 in which at least some sequences that differ for respective domain names refer to symptoms, diseases, or treatments.

33. The method of claim 31 also including
maintaining a main website having one of the domain names, and
including in the main website a link to one of the websites having another of the domain names.

34. The method of claim 31 in which websites having the respective domain names provide users of the websites with information about symptoms, diseases, or treatments associated with the symptoms, diseases, or treatments to which the different sequences of the domain names refer.

35. The method of claim 31 in which authorizing includes licensing the domain names.

36. The method of claim 31 in which authorizing includes granting sponsorship rights for the domain names.

37. The method of claim 31 in which the domain names are used for websites that comprise a network of websites.

38. The method of claim 31 also including
on at least one of the websites, providing to a user at least one of a promotional offer for a product or service offered by one of the health-related enterprises, information sheets providing printed information about a symptom, disease, or treatment, or appointment information for a healthcare provider.

39. The method of claim 31 also including
on at least one of the web sites, providing to a user at least one of a coupon for product purchase, an information sheet, or prescription information to be provided by the user to a healthcare provider for use in writing a prescription or otherwise facilitating a product purchase.

40. The method of claim 31 also including
on at least one of the web sites, providing to a healthcare provider at least one of a coupon for product purchase, an information sheet, or prescription information to be accessed by the healthcare provider for use in writing a prescription or otherwise facilitating a product purchase for a client or patient.

41. The method of claim 31 also including
on at least one of the web sites, providing to a user a sales offer for a textbook or electronic information about a symptom, disease, or treatment.

42. The method of claim 31 also including
on at least one of the web sites, providing to a user a sales offer to purchase a product associated with a symptom, disease, or treatment.

43. The method of claim 31 also including
on at least one of the web sites, providing a hyperlink to a web site of a physical store to enable a user in the physical store to use a handheld device to scan products and obtain information related to the website.

44. The method of claim 31 also including
on at least one of the web sites, providing a hyperlink to a web site that offers for sale products of at least one of the different health-related enterprises.

45. The method of claim 31 also including
on at least one of the web sites, providing a mechanism to communicate with one of the health delivery organizations.

46. The method of claim 31 also including
on at least one of the web sites, providing a mechanism to schedule appointments with one of the health delivery organizations.

47. The method of claim 31 also including
on at least one of the web sites, providing a mechanism for users to add information to a medical record.

48. The method of claim 31 also including
on at least one of the web sites, providing a mechanism for physician referrals.

49. The method of claim 31 also including
on at least one of the web sites, providing a mechanism for a user to identify himself to a health-care related enterprise or to a main one of the websites and to receive additional information or discount offers in response to identifying himself.

50. The method of claim 31 in which the displaying, enabling, and providing are done on an electronic network.

51. The method of claim 50 in which the network comprises the World Wide Web.

52. The method of claim 31 in which the displaying, enabling, and providing are done on a mobile device.

53. The method of claim 31 also including
using the websites in connection with delivery of healthcare through a physician.
